(19) United States
(12) Patent Application Publication
(10) Pub. No.: US 2024/0100008 A1
(43) Pub. Date: Mar. 28, 2024

(54) ADMINISTRATION OF GLYCEROL TRIBENZOATE AND GLYCEROL PHENYLBUTYRATE FOR TREATMENT OF NEURODEGENERATIVE AND UREA CYCLE DISORDERS

(71) Applicant: Rush University Medical Center, Chicago, IL (US)

(72) Inventor: Kalipada Pahan, Skokie, IL (US)

(21) Appl. No.: 18/257,817

(22) PCT Filed: Dec. 15, 2021
(86) PCT No.: PCT/US2021/063506

§ 371 Date: Jun. 15, 2023

Related U.S. Application Data
(60) Provisional application No. 63/126,346, filed on Dec. 16, 2020.

(51) Int. Cl. A61K 31/235; A61P 3/00; A61P 25/28

(52) U.S. Cl. CPC A61K 31/235; A61P 3/00; A61P 25/28

(57) ABSTRACT

The present disclosure generally relates to This disclosure relates to pharmaceutical compositions useful for inhibiting the progression of urea cycle disorders and neurodegenerative disorders, such as Huntington's disease, Parkinson's disease, dementia with Lewy bodies, multiple system atrophy, Alzheimer's disease, multiple sclerosis, and amyotrophic lateral sclerosis. The pharmaceutical compositions may include glycerol tribenzoate and glycerol phenylbutyrate. The pharmaceutical compositions may be administered to the patient in any suitable manner, such as intranasally or orally.

### CROSS-REFERENCE TO RELATED APPLICATIONS

The present application claims the benefit of U.S. Provisional Patent Application No. 63/126,346, filed Dec. 16, 2020, the contents of which are incorporated into the present application in their entirety.

### FIELD OF THE INVENTION

The present disclosure generally relates to pharmaceutical compositions useful for the treatment of diseases and disorders. More particularly, the disclosure relates to pharmaceutical compositions comprising glycerol tribenzoate and glycerol phenylbutyrate for the treatment of neurodegenerative and urea cycle disorders.

### BACKGROUND

Nonketotic hyperglycinemia (NKH) or glycine encephalopathy is a rare inborn error of metabolism, which is caused by the deficiency of glycine cleavage system. Most of the cases are caused by mutations in the glycine decarboxylase (GLDC) gene. Therefore, biochemically, NKH is characterized by markedly elevated level of glycine in in blood, brain, and cerebrospinal fluid. Due to such increase in glycine, NKH always exhibits complex and diverse phenotypes, such as seizures, hypotonia, cognitive impairment, developmental delays, and myoclonic jerks, ultimately leading to apnea and even death in infancy or early childhood.

Urea cycle disorders (UCDs) are disorders caused by rare inborn errors of metabolism involving mutations in the genes that encode one several enzymes or transporters necessary for normal function of the urea cycle. Affected individuals cannot remove ammonia from their body generated from protein metabolism, ultimately leading to severer cognitive deficits and brain damage. Sodium benzoate is the only available drug for glycine encephalopathy. Sodium benzoate is also one of the drugs used for UCD. However, the main problem is that sodium benzoate itself is also quickly excreted out from the body through urine. Therefore, patient is treated with sodium benzoate several times a day at high doses to maintain its effective concentration in the blood. Due to such a high dose sodium benzoate, patients feel drowsy and also suffer from nausea, vomiting and headache.

Sodium benzoate is a widely-used food preservative due to its anti-microbial properties. It also has medical importance as a component of Ucephan™, a Food and Drug Administration (FDA)-approved drug used in the treatment for hepatic metabolic defects associated with hyperammonemia, such as urea cycle disorder. The present inventor explored a novel use of sodium benzoate in treating the disease process of relapsing-remitting EAE in female SJL/J mice (see Brahmachari and Pahan, “Sodium benzoate, a food additive and a metabolite of cinnamon, modifies T cells at multiple steps and inhibits adoptive transfer of experimental allergic encephalomyelitis,” J. Immunol., 2007, Jul. 1; 179(1):275-83, the entire contents of which are expressly incorporated into the present application by reference).

The present inventor also discovered that sodium benzoate suppresses the disease process of multiple sclerosis in mice. The inventor has also discovered that sodium benzoate upregulates a protein called DJ-1, which is a beneficial, neuroprotective protein having implications in neurodegenerative disorders, such as Parkinson's disease and Alzheimer's disease (see Khasnavis and Pahan, “Sodium Benzoate, a Metabolite of Cinnamon and a Food Additive, Upregulates Neuroprotective Parkinson Disease Protein DJ-1 in Astrocytes and Neurons,” Journal of Neuroimmune Pharmacology, June 2012, Volume 7, Issue 2, pp 424-435, the entire contents of which are expressly incorporated into the present application by reference).

Further, it has been found that the level of neurotrophic factors, such as brain-derived neurotrophic factor (BDNF) and neurotrophin-3 (NT-3), decreases in the brain of patients with different neurodegenerative disorders, such as Huntington's disease, Alzheimer's disease, Parkinson's disease, and amyotrophic lateral sclerosis. Recently, the present inventor delineated that sodium benzoate increases the production of BDNF and NT-3 in brain cells, indicating that it could be beneficial for neurodegenerative disorders (see Jana et al., “Up-regulation of neurotrophic factors by cinnamon and its metabolite sodium benzoate: therapeutic implications for neurodegenerative disorders,” J. Neuroimmune Pharmacol., 2013 June; 8(3):739-55, the entire contents of which are expressly incorporated into the present application by reference).

In addition, the present inventor has shown that sodium benzoate is capable of in attenuating oxidative stress and protecting memory and learning in an animal model of Alzheimer's disease (see Modi et al., “Cinnamon and Its Metabolite Sodium Benzoate Attenuate the Activation of p21rac and Protect Memory and Learning in an Animal Model of Alzheimer's Disease”, PloS ONE, 2015, 10(6): e0130398).

However, sodium benzoate suffers from the problem that it is quickly metabolized and excreted from the body. WO2019070478, also from the current inventor, discloses the unique discovery that glyceryl dibenzoate and glyceryl (i.e. glycerol) tribenzoate provide a sustained-release and a slow-release form of sodium benzoate, which allows for a reduced administration regime and improved patient compliance in patients suffering from glycine encephalopathy. Similarly, WO2015109215, also from the current inventor, discloses the unique discovery that glyceryl dibenzoate and glyceryl tribenzoate provide a sustained-release and a slow-release form of sodium benzoate, in patients suffering from urea cycle and neurodegenerative disorders.

Glycerol phenylbutyrate (RAVICTIR), is a medication approved for use in the treatment of certain inborn urea cycle disorders and that is administered as an oral liquid.

Intranasal drug administration has been shown to offer many advantages over standard systemic delivery systems, such as its non-invasive character, a fast onset of action and in many cases reduced side effects due to a more targeted delivery. Intranasal drug delivery has been suggested to be a particularly interesting delivery route for the treatment of neurological or neurodegenerative disorders. Systemic approaches often fail to efficiently supply the CNS with drugs. See for example Keller et al., “Intranasal drug delivery: opportunities and toxicologic challenges during drug development,” (2021) Drug Delivery and Translational Research https://doi.org/10.1007/s13346-020-00891-5.

However, there are no other studies describing the intranasal use of either glycerol tribenzoate and/or glycerol phenylbutyrate. The present disclosure addresses this need.

### SUMMARY OF THE DISCLOSURE

The inventors have discovered methods and pharmaceutical compositions and/or formulations useful for the treatment of neurodegenerative and urea cycle disorders. More particularly, the present disclosure relates to methods and pharmaceutical compositions and/or formulations comprising administering to a patient in need thereof an effective amount of a pharmaceutical composition comprising glycerol tribenzoate and glycerol phenylbutyrate for the treatment of neurodegenerative and urea cycle disorders.

In some embodiments, the pharmaceutical composition is administered to the patient by injection, orally, with a transdermal patch, and/or intranasally. In some embodiments, the pharmaceutical composition is preferably inhaled by the patient.

In some embodiments, the pharmaceutical composition is administered to the patient one or more times per day and the effective amount of each active ingredient of glycerol tribenzoate and glycerol phenylbutyrate is from about 1 mg/kg body weight per day to about 200 mg/kg body weight per day of each active ingredient. For example, the composition may be administered one, two, three or more times per day.

In other embodiments, the pharmaceutical composition is formulated together with a pharmaceutically acceptable carrier or excipient. For example, in some embodiments, the pharmaceutical composition is formulated together with saline.

In still other embodiments, the pharmaceutical composition comprises from about 20 wt. % to about 99 wt. % glycerol tribenzoate and from about 20 wt. % to about 99 wt. % glycerol phenylbutyrate.

In yet other embodiments, the neurodegenerative disorder is selected from the group consisting of Huntington's disease, Parkinson's disease, dementia with Lewy bodies, multiple system atrophy, Alzheimer's disease, multiple sclerosis, amyotrophic lateral sclerosis, and any combination thereof.

In some embodiments, the method reduces a level of aggregated α-synuclein in the brain. In some embodiments, the method reduces glial cell activation.

The present disclosure also provides processes for preparing pharmaceutical compositions for the treatment of neurodegenerative disorders. The processes may comprise mixing glycerol tribenzoate and glycerol phenylbutyrate together with a pharmaceutically acceptable carrier or excipient.

In yet other embodiments, the pharmaceutical composition may comprise from about 20 wt. % to about 99 wt. % glycerol tribenzoate and from about 20 wt. % to about 99 wt. % glycerol phenylbutyrate.

The present disclosure also provides methods for inhibiting the progression of urea cycle disorders. The methods may comprise administering to a patient in need thereof an effective amount of a pharmaceutical composition comprising glycerol tribenzoate and glycerol phenylbutyrate.

The pharmaceutical composition may be administered to the patient in a manner selected from injection, inhalation, transdermally, orally, intranasally, and any combination of the foregoing. Preferably, the composition is administered to the patent intranasally. In other embodiments, the compositions are administered orally.

In some embodiments, the pharmaceutical composition is administered to the patient one time per day.

In some embodiments, the pharmaceutical composition is administered to the patient one or more times per day and the effective amount of each active ingredient of glycerol tribenzoate and glycerol phenylbutyrate is from about 1 mg/kg body weight per day to about 200 mg/kg body weight per day of each active ingredient. For example, the composition may be administered one, two, three or more times per day.

In still other embodiments, the pharmaceutical composition is formulated together with a pharmaceutically acceptable carrier or excipient, such as saline. The pharmaceutical composition may comprise from about 20 wt. % to about 99 wt. % glycerol tribenzoate and from about 20 wt. % to about 99 wt. % glycerol phenylbutyrate.

The urea cycle disorder may be selected from the group consisting of N-acetylglutamate synthase (NAGS) deficiency, Carbamoyl Phosphate Synthetase 1 (CPS1) deficiency, Ornithine transcarbamoylase (OTC) deficiency, Argininosuccinate synthase (ASS) deficiency, argininosuccinate lyase (ASL) deficiency, Arginase 1 (ARG1) deficiency, and any combination thereof. In patients with urea cycle disorders, the pharmaceutical compositions of the current invention may be administered orally.

The present disclosure also provides processes for preparing a pharmaceutical composition for the treatment of a urea cycle disorder. The processes may comprise mixing glycerol tribenzoate and glycerol phenylbutyrate together with a pharmaceutically acceptable carrier or excipient.

The pharmaceutical composition may comprise from about 20 wt. % to about 99 wI. % glycerol tribenzoate and from about 20 wt. % to about 99 wt. % glycerol phenylbutyrate. The pharmaceutically acceptable carrier may comprise saline.

In any of the embodiments described herein, doses of each of the active ingredients may be, depending upon the particular disorder being treated, for intranasal administration, glycerol tribenzoate may range from about 1 to about 10 mg/kg body weight per day and glycerol phenylbutyrate may range from about 1 to about 10 mg/kg body weight per day.

In still other embodiments described herein, doses of each of the active ingredients may be, depending upon the particular disorder being treated, for oral administration, glycerol tribenzoate may range from about 25 to about 200 mg/kg body weight per day and glycerol phenylbutyrate may range from about 25 to about 200 mg/kg body weight per day.

These and other embodiments and features of the disclosure will become more apparent through reference to the following description, the accompanying figures, and the claims. Furthermore, it is to be understood that the features of the various embodiments described herein are not mutually exclusive and can exist in various combinations and permutations.

### BRIEF DESCRIPTION OF THE DRAWINGS

FIG. TA and FIG. 1B show Western blot data of the level of α-synuclein in Triton X-100 insoluble and soluble fractions of nigra.

FIG. 2A and FIG. 2B show Western blot data of the level of α-synuclein in Triton X-100 insoluble and soluble fractions of hippocampus.

FIG. 3A and FIG. 3B show Western blot data of the levels of microglial marker Iba-1 and astroglial marker GFAP in nigral homogenates.

FIG. 4A-4C show heat map images from a Barnes maze, latency, and errors, respectively. FIG. 4D and FIG. 4E show results from a pole test.

FIG. 5A-5C show heat map images, distance, and center frequency, respectively.

FIG. 5D and FIG. 5E show results depicting rotorod latency and grip test latency, respectively.

FIG. 6A-6C show the results of oral administration of the combination of glycerol tribenzoate and glycerol phenylbutyrate improves cognitive functions in an animal model of urea cycle disorders.

### DETAILED DESCRIPTION

Throughout this disclosure, various quantities, such as amounts, sizes, dimensions, proportions, and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The present disclosure is based on the discovery that methods and pharmaceutical compositions and/or formulations comprising the intranasal administration of glycerol tribenzoate and glycerol phenylbutyrate for the treatment of neurodegenerative and urea cycle disorders has several advantages versus other previously used methods and compositions. These advantages the amount of the composition administered can be significantly decreased as compared to other modes of administration.

Although sodium benzoate exhibits beneficial effects in connection with urea cycle disorders, Huntington's disease, Parkinson's disease, multiple sclerosis, Alzheimer's disease, and other neurodegenerative disorders, the fact that the sodium benzoate is quickly metabolized and excreted from the body poses certain problems that can only be addressed by repeatedly administering this compound to patients throughout the day. Therefore, a slow-release form of sodium benzoate that allows for a reduced administration regime and improved patient compliance would be beneficial.

The present disclosure addresses this issue by providing novel treatments for urea cycle disorders and neurodegenerative disorders including, but not limited to, Huntington's disease, Parkinson's disease, dementia with Lewy bodies, multiple system atrophy, Alzheimer's disease, multiple sclerosis, amyotrophic lateral sclerosis, among others, that require only a single daily administration of a pharmaceutical composition. Moreover, the inventor discovered that by delivering the pharmaceutical composition intranasally to the patient, the amount of the composition administered can be significantly decreased as compared to other modes of administration.

The pharmaceutical compositions disclosed herein comprise glycerol tribenzoate and glycerol phenylbutyrate. Glycerol tribenzoate will slowly form sodium benzoate in the body since the molecule will be cleaved in the intestine by various lipases. Therefore, it is hypothesized that glycerol tribenzoate will exhibit highly improved therapeutic efficacies as compared to sodium benzoate. Glycerol phenylbutyrate, which slowly produces phenylbutyrate in the body upon metabolism, also exhibits highly improved therapeutic efficacies as compared to sodium phenylbutyrate.

The amount of glycerol tribenzoate and glycerol phenylbutyrate is not particularly limited. In some embodiments, the pharmaceutical composition comprises from about 20 wt. % to about 99 wt. % glycerol tribenzoate and from about 20 wt. % to about 99 wt. % glycerol phenylbutyrate. In some embodiments, the pharmaceutical composition comprises from about 25 wt. % to about 90 wt. % glycerol tribenzoate and from about 25 wt. % to about 90 wt. % glycerol phenylbutyrate. In some embodiments, the pharmaceutical composition comprises from about 30 wt. % to about 80 wt. % glycerol tribenzoate and from about 30 wt. % to about 80 wt. % glycerol phenylbutyrate. In some embodiments, the pharmaceutical composition comprises from about 35 wt. % to about 70 wt. % glycerol tribenzoate and from about 35 wt. % to about 70 wt. % glycerol phenylbutyrate. In some embodiments, the pharmaceutical composition comprises from about 40 wt. % to about 65 wt. % glycerol tribenzoate and from about 40 wt. % to about 65 wt. % glycerol phenylbutyrate. In some embodiments, the pharmaceutical composition comprises from about 40 wt. % to about 60 wt. % glycerol tribenzoate and from about 40 wt. % to about 60 wt. % glycerol phenylbutyrate. In some embodiments, the pharmaceutical composition comprises from about 45 wt. % to about 55 wt. % glycerol tribenzoate and from about 45 wt. % to about 55 wt. % glycerol phenylbutyrate.

In some embodiments of the present disclosure, a treatment is disclosed for inhibiting the progression of urea cycle disorders. A urea cycle disorder is a genetic disorder caused by a deficiency of one of the enzymes in the urea cycle that is responsible for removing ammonia from the blood stream. There are six known disorders of the urea cycle. Each can be classified by the initials of the missing enzyme. Thus, the six known urea disorders may be referred to as N-acetylglutamate synthase (NAGS) deficiency, Carbamoyl Phosphate Synthetase 1 (CPS1) deficiency, Ornithine transcarbamoylase (OTC) deficiency, Argininosuccinate synthase (ASS) deficiency, argininosuccinate lyase (ASL) deficiency, and Arginase 1 (ARG1) deficiency. The treatment comprises administering an effective amount of a pharmaceutical composition comprising glycerol tribenzoate and glycerol phenylbutyrate to a patient in need thereof. In accordance with the present disclosure, the treatment may be administered one time per day. In some aspects, the treatment may include a twice-daily administration.

The methods and compositions of the current invention are also useful for the treatment for inhibiting the progression of neurodegenerative disorders. The treatment comprises administering an effective amount of a pharmaceutical composition comprising glycerol tribenzoate and glycerol phenylbutyrate to a patient in need thereof. In some aspects, the neurodegenerative disorders may be identified by a decrease in levels of BDNF or NT-3 relative to normal subjects. In accordance with the present disclosure, the treatment may be administered one time per day. In some aspects, the treatment may be administered two times per day, three times per day, or more than three times per day.

The glycerol tribenzoate and glycerol phenylbutyrate may be formulated for administration. Methods of formulation are well known in the art (see, for example, Remington: The Science and Practice of Pharmacy, Mack Publishing Company, Easton, Pa., 19th Edition (1995)). Pharmaceutical compositions for use in accordance with the present disclosure can be in the form of sterile, non-pyrogenic liquid solutions or suspensions, coated capsules, lyophilized powders, intranasal formulations or other forms known in the art.

Pharmaceutically Acceptable Carrier

As used herein, the term “pharmaceutically acceptable carrier” means a non-toxic, inert solid, semi-solid or liquid filler, diluent, encapsulating material or formulation auxiliary of any type. In the treatment methods contemplated by the present disclosure, the glycerol tribenzoate and glycerol phenylbutyrate may be used alone or in compositions together with a pharmaceutically acceptable carrier or excipient, such as saline. For example, an oral dosage form composition may comprise glycerol tribenzoate and glycerol phenylbutyrate in addition to a pharmaceutically acceptable carrier. An inhalation dosage form composition may comprise glycerol tribenzoate and glycerol phenylbutyrate in addition to a pharmaceutically acceptable carrier. A composition for buccal administration may comprise glycerol tribenzoate and glycerol phenylbutyrate in addition to a pharmaceutically acceptable carrier. A composition for nasal administration may comprise glycerol tribenzoate and glycerol phenylbutyrate in addition to a pharmaceutically acceptable carrier. Further, if a transdermal patch is used as the method of administering the glycerol tribenzoate and glycerol phenylbutyrate to the patient, the transdermal patch may comprise the glycerol tribenzoate and glycerol phenylbutyrate in addition to a pharmaceutical acceptable carrier.

Some examples of materials which can serve as pharmaceutically acceptable carriers are sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil; safflower oil; sesame oil; olive oil; corn oil and soybean oil; glycols, such as propylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol, and phosphate buffer solutions, as well as other non-toxic compatible lubricants, such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, the contents of which are expressly incorporated herein by reference.

Oral Dosage Forms

In certain embodiments, the glycerol tribenzoate and glycerol phenylbutyrate may be orally administered to be ingested by humans and other animals. Solid dosage forms for oral administration include, as illustrative but non-limiting examples, capsules, tablets, pills, powders, thin films and granules. In solid dosage forms, the active compound may be mixed with at least one inert, pharmaceutically acceptable excipient or carrier, as described in more detail below.

As illustrative, non-limiting examples, an oral dosage form of the presently disclosed pharmaceutical composition may be mixed with about 0.1% to about 1%, such as about 0.5%, methyl cellulose. A pharmaceutical composition according to the present disclosure for intranasal administration may be mixed with about 1 to about 10 μl, such as about 5 μl, of saline. A pharmaceutical composition according to the present disclosure for nebulization may be solubilized in about 100 to about 300 μl saline, such as about 200 μl saline.

Stabilizers

A composition, formulation, or dosage form herein may further comprise one or more glycerol tribenzoate and/or glycerol phenylbutyrate stabilizers. As used herein, a glycerol tribenzoate or glycerol phenylbutyrate stabilizer is a substance that extends the time before which the glycerol tribenzoate or glycerol phenylbutyrate is converted to a salt in the environment in which the formulation or dosage form is administered, in comparison to the conversion in its absence. Non-limiting examples of stabilizers include phosphatidyl choline, phosphatidyl inositol, phosphatidyl ethanolamine, or other phospholipids. A composition, formulation, or dosage form further comprising one or more stabilizers may be administered in any one of the methods herein. A glycerol phenylbutyrate and/or glycerol tribenzoate stabilizer may be present in an amount of about 50 mg to about 1000 mg in a composition, formulation, or dosage form herein. In some embodiments, the stabilizer may be present in an amount ranging from about 50 mg to about 500 mg or about 50 mg to about 100 mg.

For example, in addition to glycerol tribenzoate, glycerol phenylbutyrate, and/or a pharmaceutically acceptable carrier, an oral dosage form composition may comprise one or more stabilizers. A stabilizer in an oral dosage from may be present in an amount of about 50 mg to about 1000 mg. In some embodiments, a stabilizer is present in an amount less than about 50 mg. In some embodiments, the stabilizer may be present in an amount ranging from about 50 mg to about 500 mg or about 50 mg to about 100 mg.

As an additional example, in addition to glycerol tribenzoate, glycerol phenylbutyrate, and/or a pharmaceutically acceptable carrier, an inhalation dosage form composition may comprise one or more stabilizers. A stabilizer in an inhalation dosage from may be present in an amount of about 50 mg to about 1000 mg. In some embodiments, the stabilizer may be present in an amount ranging from about 50 mg to about 500 mg, about 50 mg to about 100 mg, or less than about 50 mg.

As a further example, in addition to glycerol tribenzoate, glycerol phenylbutyrate, and/or a pharmaceutically acceptable carrier, a composition for buccal administration may comprise one or more stabilizers. A stabilizer in a composition for buccal administration may be present in an amount of about 50 mg to about 1000 mg. In some embodiments, the stabilizer may be present in an amount ranging from about 50 mg to about 500 mg, about 50 mg to about 100 mg, or less than about 50 mg.

In addition to glycerol tribenzoate, glycerol phenylbutyrate, and/or a pharmaceutically acceptable carrier, a transdermal patch may comprise one or more stabilizers. A stabilizer in a composition for transdermal administration may be present in an amount of about 50 mg to about 1000 mg. In some embodiments, the stabilizer may be present in an amount ranging from about 50 mg to about 500 mg, about 50 mg to about 100 mg, or less than about 50 mg. As is commonly understood in the art, a transdermal patch is an adhesive patch that is placed on the skin of a patient. The patch comprises a composition/medication and delivers the composition/medication to the patient through the skin.

Intranasal Compositions

In some embodiments, the pharmaceutical composition may be administered to a patient as nasal drop (intranasally) or using a nebulization technique. A nebulizer may be used to change a liquid solution of a pharmaceutical composition into a fine mist that may be inhaled by a patient. The inventor determined numerous benefits of these techniques.

For example, the dosage of the pharmaceutical composition can be significantly decreased when either nasal drop or nebulization is used as the delivery method. In some instances, the dosage may be reduced by about one tenth or one twentieth as compared to, for example, injections, oral administration/ingestion of a liquid solution or oral administration/ingestion of a pill. Moreover, using a nebulization technique or nasal drop bypasses the digestive system whereas ingesting a pill or liquid solution of a pharmaceutical composition sends the composition to the digestive system. For example, diarrhea is a common side effect in some urea cycle disorder patients taking glycerol phenylbutyrate orally. Such side effect will be avoided by intranasal administration of glycerol phenylbutyrate and glycerol tribenzoate. Finally, using either a nasal drop or nebulization technique allows the pharmaceutical composition to travel from the olfactory bulb directly to the brain.

In some embodiments, the nebulized pharmaceutical composition may be inhaled through one or both of the mouth or the nasal passage. Without being bound to any theory, it is believed that nasal administration of the composition can take advantage of “nose-to-brain” (N2B) transport systems in which several possibilities exist for bypassing the blood-brain-barrier for direct delivery to the brain. These include the draining of drugs absorbed in the nasal mucosa into the sinus and eventually to the carotid artery, where a “counter-current transfer” from venous blood to the brain may occur. Lymphatic drainage into the perivascular space from the olfactory trigeminal nerves between the central nervous system (CNS) have also been postulated as the mechanism of N2B transport.

Nebulizers are known in the art and the invention of the present disclosure can be used in connection with any nebulizer. For example, the pharmaceutical composition disclosed herein may be nebulized with an inhaler or a Buxco© Inhalation Tower All-In-One Controller.

Excipients

Illustrative, non-limiting examples of excipients or carriers include sodium citrate or dicalcium phosphate and/or a) one or more fillers or extenders (a filler or extender may be, but is not limited to, one or more selected from starches, lactose, sucrose, glucose, mannitol, and silicic acid), b) one or more binders (binders may be selected from, but not limited to, carboxymethylcellulose, alginates, gelatin, polyvinylpyrrolidinone, sucrose, and acacia), c) one or more humectants (a humectant may be, but is not limited to, glycerol), d) one or more disintegrating agents (disintegrating agents may be selected from, but are not limited to, agar-agar, calcium carbonate, potato or tapioca starch, alginic acid, silicates, and sodium carbonate), e) one or more solution retarding agents (for example, but not limited to, paraffin), f) one or more absorption accelerators (selected from, but not limited to, quaternary ammonium compounds), g) one or more wetting agents (for example, but not limited to, acetyl alcohol and glycerol monostearate), h) one or more absorbents (selected from, but not limited to, kaolin and bentonite clay), and i) one or more lubricants (selected from, but not limited to, talc, calcium stearate, magnesium stearate, solid polyethylene glycols, and sodium lauryl sulfate). In the case of capsules, tablets and pills, for example, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells. Illustrative, non-limiting examples of coatings and shells include enteric coatings and other coatings/shells well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that may be used include, but are not limited to, polymeric substances and waxes.

The active compounds can also be in micro-encapsulated form with one or more excipients as noted above. The solid dosage forms of tablets, dragees, capsules, pills, and granules can be prepared with coatings and shells. The coatings or shells may be, but are not limited to, enteric coatings, release-controlling coatings and other coatings in the pharmaceutical formulating art. In solid dosage forms, the active compound may be admixed with at least one inert diluent. The inert diluent may include, but is not limited to, one or more of, sucrose, lactose or starch. Dosage forms may also comprise additional substances other than inert diluents. The additional substances may be, but are not limited to, tableting lubricants and other tableting aids. The tableting lubricants and other aids may be, but are not limited to, magnesium stearate and microcrystalline cellulose. In the case of capsules, tablets and pills, for example, the dosage forms may also comprise buffering agents. They may comprise opacifying agents. They may be of a composition that releases the active ingredient(s) only, or preferentially, in a certain part of the intestinal tract. The release may be in a delayed manner. Examples of embedding compositions that can be used include, but are not limited to, polymeric substances and waxes.

Liquid Dosage Forms

Liquid dosage forms for oral administration include, but are not limited to, pharmaceutically acceptable emulsions, microemulsions, solutions, suspensions, syrups and elixirs. In addition to the active compounds, the liquid dosage forms may comprise one or more inert diluents. The inert diluents may be selected from those commonly used in the art. Illustrative, non-limiting examples of inert diluents include water or other solvents, solubilizing agents and emulsifiers including, but not limited to, ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. The oral compositions may comprise one or more adjuvants. Illustrative, non-limiting examples of adjuvants include wetting agents, emulsifying and suspending agents, sweetening, flavoring, and perfuming agents.

The amount of carrier in a composition disclosed herein is not particularly limited. As an example, for a liquid oral treatment composition, the composition may comprise from about 0.1% carrier to about 1% carrier, such as about 0.5% methyl cellulose. In some embodiments, for intranasal administration, the composition may comprise from about 1 μl to about 10 μl of the carrier, such as about 5 μl saline. In some embodiments, for nebulization, the composition may comprise from about 50 μl to about 500 μl of the carrier, such as about 100 μl, about 200 μl or about 300 μl saline.

“Effective or Therapeutic Amount”

Effective or therapeutic amounts of the compositions of this disclosure include any amount sufficient to inhibit (e.g., slow or stop) the progression of a urea cycle disorder and/or a neurodegenerative disorder. In some embodiments, effective amounts of the compositions include any amount sufficient to inhibit (e.g., slow or stop) the deterioration of a locomotor activity of a patient. In some embodiments, effective amounts of the compositions include any amount sufficient to improve a locomotor activity of a patient. In some embodiments, effective amounts of the compositions include any amount sufficient to reduce a level of aggregated α-synuclein in the brain. In some embodiments, effective amounts of the compositions include any amount sufficient to reduce glial cell activation.

The amount of active ingredient (glycerol tribenzoate and glycerol phenylbutyrate) that may be combined with the optional carrier materials to produce a single dosage form may vary depending upon the host treated and the particular mode of administration. The specific dose level for any particular patient may depend upon a variety of factors including the activity of the specific compound employed, the age, body weight, general health, sex, diet, time of administration, route of administration, rate of excretion, drug combination, and the severity of the particular disorder or disease undergoing therapy. A therapeutically effective amount for a given situation can be readily determined by routine experimentation and is within the skill and judgment of the ordinary clinician.

In accordance with certain methods of treatment disclosed in the present application, progression of various disorders is slowed or stopped in a patient (a patient may be a human, a lower mammal, or a warm-blooded animal), by administering to the patient an effective amount of the glycerol tribenzoate and glycerol phenylbutyrate in such amounts, and for such time as is necessary, to achieve the desired result. An amount of a compound that is effective to slow or stop the progression of a disease or disorder may refer to a sufficient amount of the compound to treat the disease or disorder at a reasonable benefit/risk ratio applicable to any medical treatment.

The total daily usage of the compounds and compositions of the present disclosure may be decided by the attending physician within the scope of sound medical judgment. The specific therapeutically effective dose level for any particular patient may depend upon a variety of factors including the disease or disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; and drugs used in combination or coincidental with the specific compound employed.

The “effective amount” or dose of a compound of the present disclosure, such as glycerol tribenzoate and glycerol phenylbutyrate, to be administered to warm-blooded animals (e.g., humans) may vary depending upon the disorder to be treated. In connection with certain urea cycle and neurodegenerative disorders, the effective amount of each active ingredient may be from approximately about 1 mg/kg body weight per day to about 200 mg/kg body weight per day of each of glycerol tribenzoate and glycerol phenylbutyrate.

However, if intranasal administration is used as the method of administering the pharmaceutical composition, the inventor determined that the aforementioned effective amounts can be significantly decreased. For example, in some embodiments, the amount administered to the patient may be from about 1 mg/kg body weight per day to about 25 mg/kg body weight per day. In some embodiments, the effective amount may be from about 1 mg/kg body weight per day to about 15 mg/kg body weight per day, from about 1 mg/kg body weight per day to about 10 mg/kg body weight per day, from about 3 mg/kg body weight per day to about 7 mg/kg body weight per day, from about 3 mg/kg body weight per day to about 5 mg/kg body weight per day, from about 2 mg/kg body weight per day to about 7 mg/kg body weight per day, or from about 2 mg/kg body weight per day to about 5 mg/kg body weight per day. In some embodiments, the amount is about 2, about 3, about 4, about 5, about 6, or about 7 mg/kg body weight per day. The administration may be once per day, twice per day, or more than two times per day.

In an embodiment, the dose of glycerol tribenzoate and glycerol phenylbutyrate delivered by oral administration is about 1 to about 200 mg/kg/day. Formulations and methods including this dose are contemplated.

In an embodiment, the dose of glycerol tribenzoate and glycerol phenylbutyrate administered by transdermal patch is about 25 to about 100 mg/kg/day. Formulations, transdermal patches including said formulations, and methods including this dose are contemplated.

In an embodiment, the dose of glycerol tribenzoate and glycerol phenylbutyrate administered by buccal administration is about 0.5 to about 5 mg/day. Formulation and methods including this dose are contemplated. The dose may be any value from about 0.5 to about 5 mg/kg/day, or in a subrange selected from any two 0.1 mg/kg/day increments from about 0.5 to about 5 mg/kg/day.

Additionally, in some embodiments, a patient may receive the glycerol tribenzoate and glycerol phenylbutyrate by multiple administration methods. In some embodiments, the glycerol tribenzoate and glycerol phenylbutyrate may be administered to the patient by injection, nebulization, buccal administration, oral administration (e.g., solution, tablet, thin film, etc.), transdermal patch, intranasally, and any combination of the foregoing. For example, the glycerol tribenzoate and glycerol phenylbutyrate may be administered to the patient intranasally in addition to an oral administration. In some embodiments, oral administration may be used to maintain an optimal drug concentration in the patient during intranasal treatment. In some embodiments, the glycerol tribenzoate and glycerol phenylbutyrate may be administered to the patient intranasally in addition to injection(s). In some embodiments, the glycerol tribenzoate and glycerol phenylbutyrate may be administered to the patient intranasally in addition to a transdermal patch. In some embodiments, the glycerol tribenzoate and glycerol phenylbutyrate may be administered to the patient intranasally in addition to using a nebulization technique. In some embodiments, the agents are administered orally only. The present disclosure encompasses any combination of the administration techniques described or contemplated herein.

The present inventor discovered that the pharmaceutical compositions disclosed herein, along with the administration methods, can be used to improve locomotor and cognitive activities (see Examples disclosed herein). As such, the present disclosure is also directed to compositions and methods useful for improving locomotor and/or cognitive activities. In some embodiments, the locomotor activities are selected from the group consisting of walking, running, jumping, and any combination thereof.

Any or all of these locomotor activities may be improved by administering a pharmaceutical composition to a patient, wherein the composition comprises glycerol phenylbutyrate and glycerol tribenzoate. In some embodiments, the composition is administered intranasally. Depending upon the administration method and the number of administrations per day (optionally among other factors), an effective amount can be selected by one of ordinary skill in the art with the guidance provided in the present application.

Additionally, the present inventor discovered that the pharmaceutical compositions disclosed herein, along with the administration methods, can be used to reduce activation of certain cells in the brain. For example, using the pharmaceutical compositions disclosed in the present application in combination with one or more of the administration methods disclosed herein, the inventor discovered that it is possible to reduce activation of astroglial cells and microglial cells in the brain (see Examples disclosed herein).

Still further, the inventor discovered that the presently disclosed pharmaceutical compositions and methods of administration can be used to reduce levels of α-synuclein in the brain (see Examples disclosed herein).

Further reference is made to the following experimental examples.

### EXAMPLES

The following examples are given for the purpose of illustrating various embodiments of the invention and are not meant to limit the present disclosure in any fashion. The present examples, along with the methods described herein are presently representative of preferred embodiments, are provided only as examples, and are not intended as limitations on the scope of the invention. Changes therein and other uses which are encompassed within the spirit of the disclosure as defined by the scope of the claims will occur to those skilled in the art.

### Example 1

Intranasal Treatment of A53T Mice with Glycerol Phenylbutyrate and Glycerol Tribenzoate Reduces the Level of Aggregated α-Synuclein in Substantia Nigra and Hippocampus

Alpha (α)-synucleinopathy is a hallmark of Parkinson's disease, dementia with Lewy bodies, and multiple system atrophy. Nine-month-old A53T Tg mice (n=4 per group) were treated intranasally with glycerol phenylbutyrate (about 5 mg/kg body wt/day) and glycerol tribenzoate (about 5 mg/kg body wt/day) in a total volume of about 5 μl vehicle (comprising saline) (about 2.5 μl in each nostril). Control A53T mice received only 5 μl vehicle intranasally.

After one month of daily intranasal treatment, the level of α-synuclein was monitored in Triton X-100 insoluble (FIG. 1A) and Triton X-100 soluble (FIG. 1B) fractions of substantia nigra by Western blot using anti-α-synuclein antibody. Actin was run as a loading control. The glycerol phenylbutyrate and glycerol tribenzoate combination markedly reduced the level of aggregated α-synuclein in Triton X-100 insoluble nigral fractions of aged A53T mice (FIG. TA). This result was specific as the intranasal glycerol phenylbutyrate and glycerol tribenzoate combination did not alter the level of soluble α-synuclein in Triton X-100 soluble nigral fractions (FIG. 1B).

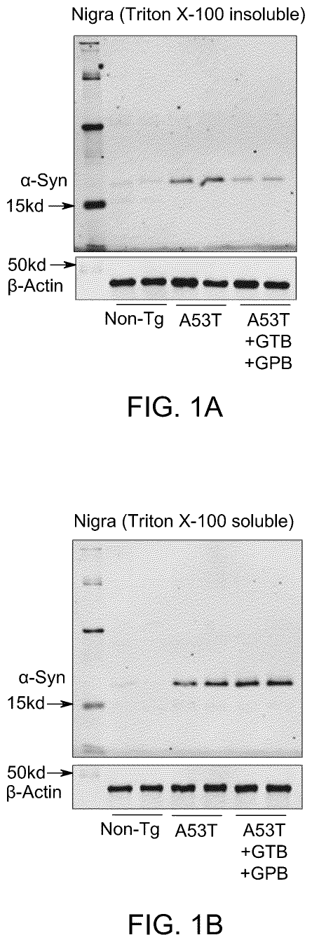

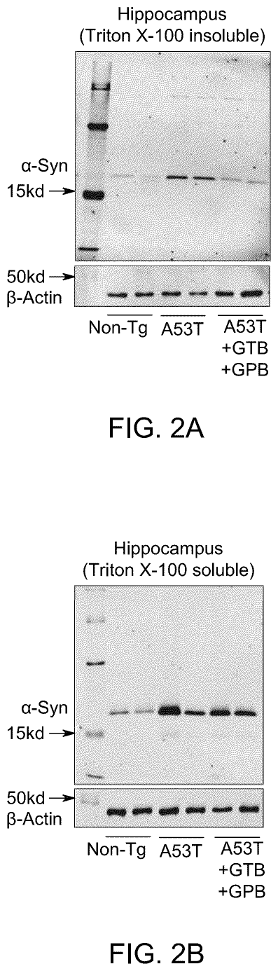

Similar to substantia nigra, the glycerol phenylbutyrate and glycerol tribenzoate combination also reduced the level of α-synuclein in Triton X-100 insoluble (FIG. 2A) but not soluble (FIG. 2B), hippocampal fractions of aged A53T mice.

### Example 2

Intranasal Treatment of A53T Mice with Glycerol Phenylbutyrate and Glycerol Tribenzoate Reduces the Glial Activation in Substantia Nigra

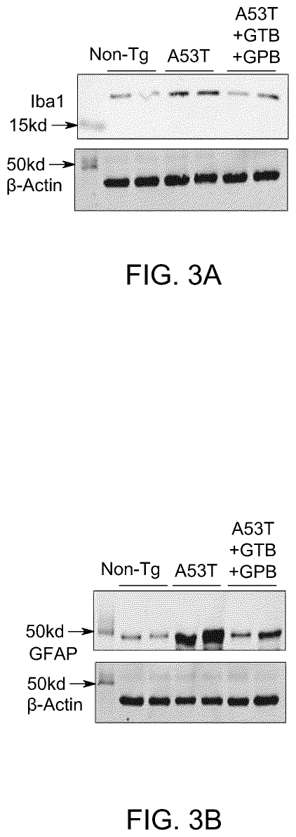

Glial activation is a hallmark of different neurodegenerative disorders including Huntington's disease, Alzheimer's disease, Parkinson's disease, multiple sclerosis, amyotrophic lateral sclerosis, multiple system atrophy, and dementia with Lewy bodies. While ionized calcium binding adaptor molecule 1 (Iba-1) is a marker of microglia, glial fibrillary acidic protein (GFAP) is considered as a prototype marker of astrocytes. Using the same experimental procedure outlined above, the inventor found increased expression of both Iba-1 and GFAP in the nigra of aged A53T mice. However, after intranasal administration, the glycerol phenylbutyrate and glycerol tribenzoate combination markedly reduced the expression of both Iba-1 (FIG. 3A) and GFAP (FIG. 3B) in the nigra of aged A53T mice.

### Example 3

Intranasal Treatment of A53T Mice with Glycerol Phenylbutyrate and Glycerol Tribenzoate Improves Motor Function and Memory and Learning Deficits

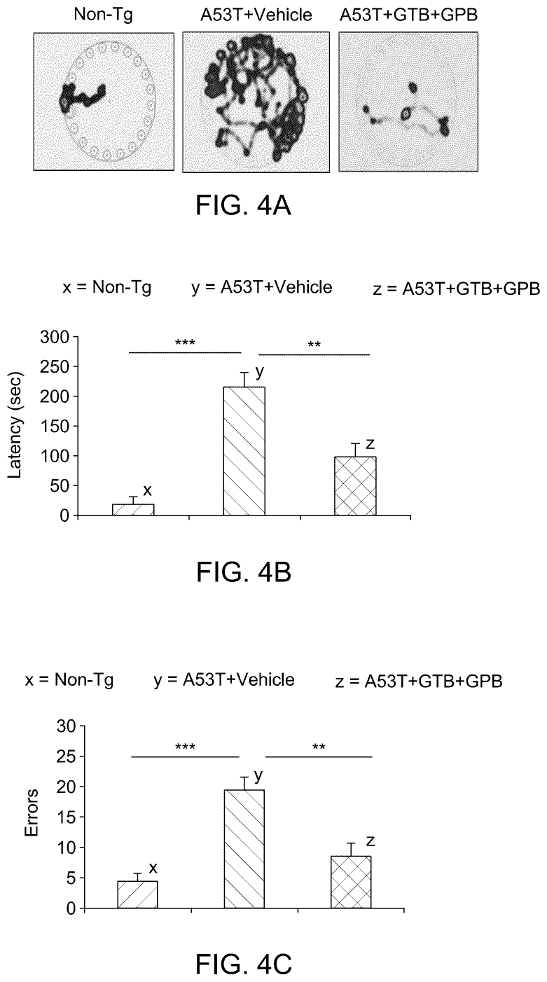

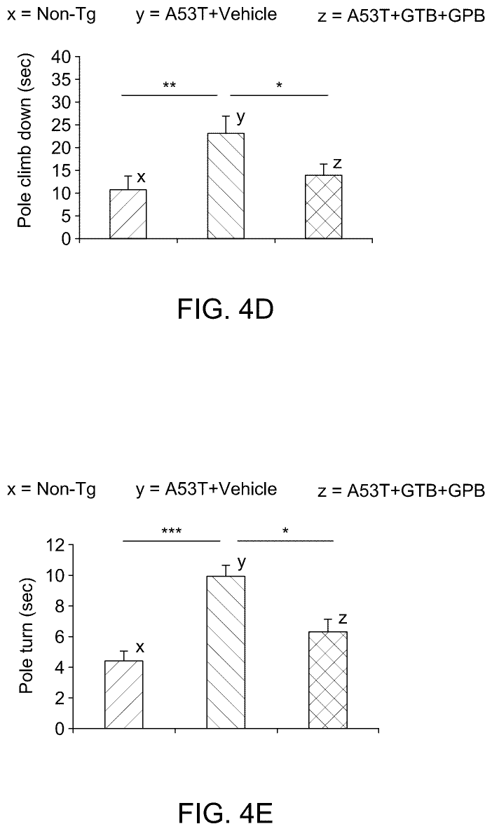

Finally, hippocampal function and motor performance of these animals were examined to check the functional outcome of inhibition of α-synucleinopathy and glial activation achieved by intranasal administration of glycerol phenylbutyrate and glycerol tribenzoate. The same procedural steps outlined above were used for these tests. After one month of treatment, memory and learning were examined by a Barnes maze. FIG. 4A depicts heat map images from a Barnes maze recorded with a Noldus camera and visualized using EthoVision XT software. FIG. 4B shows latency to find the target hole and FIG. 4C depicts the number of errors.

Locomotor activity was monitored by a pole test. FIG. 4D shows time to climb down the pole and FIG. 4E shows the pole turn time.

Marked impairment in spatial memory and learning in A53T Tg mice as compared to non-TG mice and improvement in such hippocampal functions by intranasal glycerol phenylbutyrate and glycerol tribenzoate administration suggests that the intranasal administration of the combination of glycerol phenylbutyrate and glycerol tribenzoate is capable of protecting memory and learning in A53T model of α-synucleinopathy. Intranasal administration of glycerol phenylbutyrate and glycerol tribenzoate also improved the performance of aged A53T mice on the pole test.

These results suggest that intranasal, low doses of the glycerol phenylbutyrate and glycerol tribenzoate combination may have therapeutic implications for at least three types of α-synucleinopathy (Parkinson's disease, dementia with Lewy bodies and multiple system atrophy). Furthermore, by attenuating glial activation, nasally administered low-dose combination of glycerol phenylbutyrate and glycerol tribenzoate may find therapeutic importance in Huntington's disease, multiple sclerosis, amyotrophic lateral sclerosis, and Alzheimer's disease.

Moreover, impairment in memory and learning is a signature characteristic feature of Alzheimer's disease, the most common neurodegenerative disease in humans. Therefore, intranasal administration of glycerol phenylbutyrate and glycerol tribenzoate may also have therapeutic importance for Alzheimer's disease.

### Example 4

Intranasal Administration of Glycerol Phenylbutyrate and Glycerol Tribenzoate in a Transgenic Mouse Model of Huntington's Disease

The inventor also investigated the effect of intranasal administration of glycerol phenylbutyrate and glycerol tribenzoate in a transgenic mouse model of Huntington's disease. Many features of Huntington's disease are modeled in N171-82Q transgenic (Tg) mice. Two-month-old N171-82Q Tg mice (n=4 per group) were treated intranasally with glycerol phenylbutyrate (about 5 mg/kg body wt/day) and glycerol tribenzoate (about 5 mg/kg body wt/day) in a total volume of about 5 μl vehicle (about 2.5 μl in each nostril) for one month. Control mice received only 5 μl vehicle intranasally. Since the ultimate goal of neuroprotection in Huntington's disease is improvement in locomotor activities, the inventor monitored open-field behavior, rotorod and grip test.

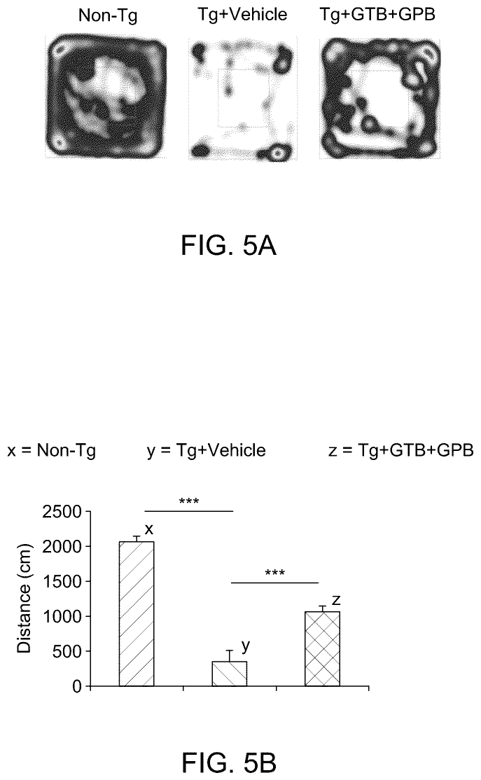

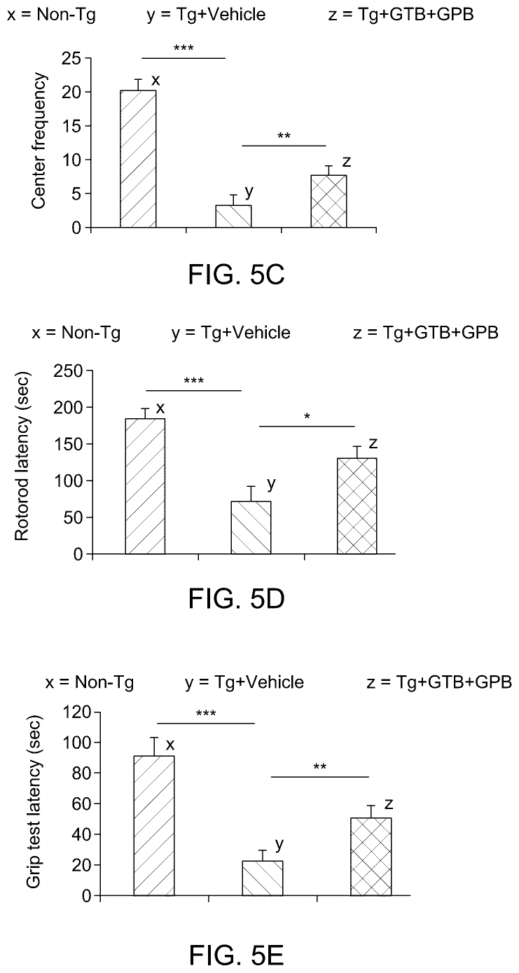

Marked impairment in open-field behaviors (FIG. 5A heat map images, FIG. 5B distance, FIG. 5C center frequency), rotorod activity/latency (FIG. 5D) and grip test latency (FIG. 5E) was observed in N171-82Q Tg mice as compared to non-Tg mice. However, after intranasal administration of the combination of low-dose glycerol tribenzoate and glycerol phenylbutyrate, the inventor observed significant improvement in locomotor activities.

These findings suggest that intranasal administration of the combination of glycerol tribenzoate and glycerol phenylbutyrate may be a therapy for Huntington's disease.

### Example 5

Oral Administration of the Combination of Glycerol Tribenzoate and Glycerol Phenylbutyrate Improves Cognitive Functions in an Animal Model of Urea Cycle Disorders

Urea cycle disorders (UCDs) are rare inborn errors of metabolism involving mutations in the genes that encode one of six enzymes or two transporters necessary for normal function of the urea cycle. Therefore, UCDs are characterized by hyperammonemia and life-threatening hyperammonemic crises. Moreover, cognitive deficits are common in individuals with inherited urea cycle disorders.

To examine this, oral administration of the combination of glycerol tribenzoate and glycerol phenylbutyrate were tested on the improvement of memory and learning in ornithine transcarbamylase (OTC) knockout mice, an animal model of UCD. OTC knockout B6EiC3Sn a/A-Otc^{spf-ash}/J(Otc^{spf-ash}) mice were purchased from the Jackson Lab.

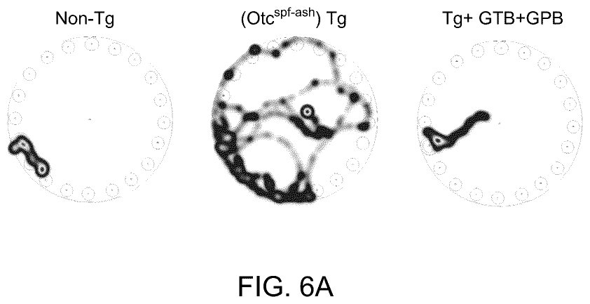

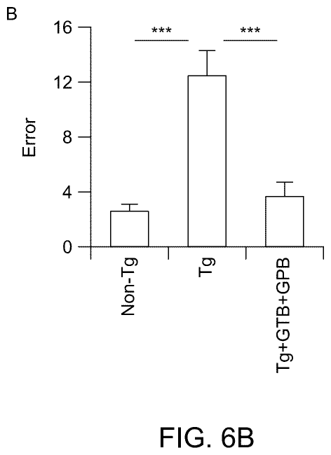

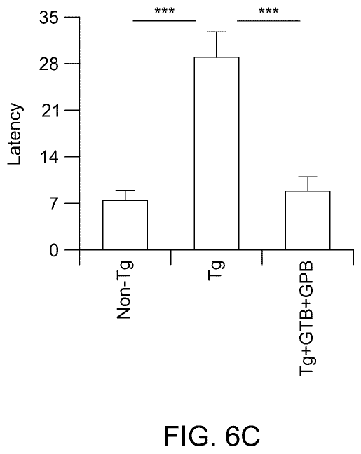

Eight-week old B6EiC3Sn a/A-Otc^{sPf-ash}/J(Otc^{spf-ash}) mice were treated with the combination of glycerol tribenzoate (50 mg/kg body weight/d) and glycerol phenylbutyrate (50 mg/kg body weight/d) orally via gavage for 10 days followed by monitoring cognitive functions by Barnes maze (FIG. 6A, representative track plots summarizing the overall activity of mice on the apparatus recorded with a Noldus camera and visualized by Ethovision XT software; FIG. 6B, error; FIG. 6C, latency). Results are mean±SEM of three mice per group. ***p<0.001.

The results demonstrate that oral administration of the combination of glycerol tribenzoate and glycerol phenylbutyrate improves memory and learning in an animal model of urea cycle disorders.

As will be appreciated from the descriptions herein, a wide variety of aspects and embodiments are contemplated by the present disclosure, examples of which include, without limitation, the aspects and embodiments listed below:

Methods and pharmaceutical compositions and/or formulations comprising administering to a patient in need thereof an effective amount of a pharmaceutical composition comprising glycerol tribenzoate and glycerol phenylbutyrate for the treatment of neurodegenerative and urea cycle disorders.

Methods wherein the pharmaceutical composition is administered to the patient by injection, orally, with a transdermal patch, and/or intranasally. Preferably, methods are provided wherein the pharmaceutical composition is inhaled by the patient.

Methods wherein the pharmaceutical composition is administered to the patient one or more times per day and the effective amount of each active ingredient of glycerol tribenzoate and glycerol phenylbutyrate is from about 1 mg/kg body weight per day to about 200 mg/kg body weight per day of each active ingredient. For example, the composition may be administered one, two, three or more times per day.

Methods wherein the pharmaceutical composition is formulated together with a pharmaceutically acceptable carrier or excipient. For example, in some embodiments, the pharmaceutical composition is formulated together with saline.

Methods wherein the pharmaceutical composition comprises from about 20 wt. % to about 99 wt. % glycerol tribenzoate and from about 20 wt. % to about 99 wt. % glycerol phenylbutyrate.

Methods wherein the neurodegenerative disorder is selected from the group consisting of Huntington's disease, Parkinson's disease, dementia with Lewy bodies, multiple system atrophy, Alzheimer's disease, multiple sclerosis, amyotrophic lateral sclerosis, and any combination thereof. The urea cycle disorder may be selected from the group consisting of N-acetylglutamate synthase (NAGS) deficiency, Carbamoyl Phosphate Synthetase 1 (CPS1) deficiency, Ornithine transcarbamoylase (OTC) deficiency, Argininosuccinate synthase (ASS) deficiency, argininosuccinate lyase (ASL) deficiency, Arginase 1 (ARG1) deficiency, and any combination thereof.

Methods wherein a level of aggregated α-synuclein is reduced in the brain. In some embodiments, the method reduces glial cell activation.

Methods for processes for preparing pharmaceutical compositions for the treatment of neurodegenerative disorders. The processes may comprise mixing glycerol tribenzoate and glycerol phenylbutyrate together with a pharmaceutically acceptable carrier or excipient.

Methods for processes for preparing pharmaceutical compositions wherein the pharmaceutical composition may comprise from about 20 wt. % to about 99 wt. % glycerol tribenzoate and from about 20 wt. % to about 99 wt. % glycerol phenylbutyrate.

Methods for inhibiting the progression of urea cycle disorders. The methods may comprise administering to a patient in need thereof an effective amount of a pharmaceutical composition comprising glycerol tribenzoate and glycerol phenylbutyrate. The pharmaceutical composition may be administered to the patient in a manner selected from injection, inhalation, transdermally, orally, intranasally, and any combination of the foregoing. Preferably, the composition is administered to the patent intranasally.

In some embodiments, the pharmaceutical composition is administered to the patient one or more times per day. In some embodiments, the effective amount is from about effective amount of each active ingredient of glycerol tribenzoate and glycerol phenylbutyrate is from about 1 mg/kg body weight per day to about 200 mg/kg body weight per day of each active ingredient. For example, the composition may be administered one, two, three or more times per day.

Methods and compositions wherein the pharmaceutical composition is formulated together with a pharmaceutically acceptable carrier or excipient, such as saline. The pharmaceutical composition may comprise from about 20 wt. % to about 99 wt. % glycerol tribenzoate and from about 20 wt. % to about 99 wt. % glycerol phenylbutyrate.

Methods are provided for processes for preparing a pharmaceutical composition for the treatment of a urea cycle disorder. The processes may comprise mixing glycerol tribenzoate and glycerol phenylbutyrate together with a pharmaceutically acceptable carrier or excipient. The pharmaceutical composition may comprise from about 20 wt. % to about 99 wt. % glycerol tribenzoate and from about 20 wt. % to about 99 wt. % glycerol phenylbutyrate. The pharmaceutically acceptable carrier may comprise saline.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for inhibiting the progression of a neurodegenerative disorder, comprising: administering to a patient in need thereof an effective amount of a pharmaceutical composition comprising glycerol tribenzoate and glycerol phenylbutyrate.

2. The method of claim 1, wherein the pharmaceutical composition is administered to the patient in a manner selected from one or more of injection, inhalation, transdermally, orally, and intranasally.

3. The method of claim 1, wherein the pharmaceutical composition is administered intranasally.

4. The method of any one of claim 1, wherein the pharmaceutical composition is administered to the patient one or more times per day.

5. The method of any one of claim 1, wherein the effective amount is from about 1 mg/kg body weight per day to about 200 mg/kg body weight per day of each of glycerol tribenzoate and glycerol phenylbutyrate.

6. The method of claim 1, wherein the pharmaceutical composition is formulated together with a pharmaceutically acceptable carrier or excipient.

7. The method of claim 1, wherein the pharmaceutical composition comprises from about 20 wt. % to about 99 wt. % glycerol tribenzoate and from about 20 wt. % to about 99 wt. % glycerol phenylbutyrate.

8. The method of claim 1, wherein the neurodegenerative disorder is selected from the group consisting of Huntington's disease, Parkinson's disease, dementia with Lewy bodies, multiple system atrophy, Alzheimer's disease, multiple sclerosis, amyotrophic lateral sclerosis, and any combination thereof.

9. The method of any one of claim 1, wherein the method reduces a level of aggregated α-synuclein in a brain.

10. The method of any one of claim 1, wherein the method reduces glial cell activation.

11. A process of preparing a pharmaceutical composition for the treatment of a neurodegenerative disorder, comprising: mixing glycerol tribenzoate and glycerol phenylbutyrate together with a pharmaceutically acceptable carrier or excipient.

12. The process of claim 11, wherein the neurodegenerative disorder is selected from the group consisting of Huntington's disease, Parkinson's disease, dementia with Lewy bodies, multiple system atrophy, Alzheimer's disease, multiple sclerosis, amyotrophic lateral sclerosis, and any combination thereof.

13. The process of claim 11, wherein the pharmaceutical composition comprises from about 20 wt. % to about 99 wt. % glycerol tribenzoate and from about 20 wt. % to about 99 wt. % glycerol phenylbutyrate.

14. The process of claim 11, wherein the pharmaceutically acceptable carrier comprises saline.

15. A method for inhibiting the progression of a urea cycle disorder, comprising: administering to a patient in need thereof an effective amount of a pharmaceutical composition comprising glycerol tribenzoate and glycerol phenylbutyrate.

16. The method of claim 15, wherein the pharmaceutical composition is administered to the patient in a manner selected from one or more of injection, inhalation, transdermally, orally, and intranasally.

17. The method of claim 16, wherein the pharmaceutical composition is administered intranasally.

18. The method of any one of claim, wherein the pharmaceutical composition is administered to the patient one or more times per day.

19. The method of claim 15, wherein the effective amount is from about 1 mg/kg body weight per day to about 200 mg/kg body weight per day of each of glycerol tribenzoate and glycerol phenylbutyrate.

20. The method of claim 15, wherein the pharmaceutical composition is formulated together with a pharmaceutically acceptable carrier or excipient.

21. The method of claim 15, wherein the pharmaceutical composition comprises from about 20 wt. % to about 99 wt. % glycerol tribenzoate and from about 20 wt. % to about 99 wt. % glycerol phenylbutyrate.

22. The method of claim 15, wherein the urea cycle disorder is selected from the group consisting of N-acetylglutamate synthase (NAGS) deficiency, Carbamoyl Phosphate Synthetase 1 (CPS1) deficiency, Ornithine transcarbamoylase (OTC) deficiency, Argininosuccinate synthase (ASS) deficiency, argininosuccinate lyase (ASL) deficiency, Arginase 1 (ARG1) deficiency, and any combination thereof.

23. A process of preparing a pharmaceutical composition for the treatment of a urea cycle disorder, comprising: mixing glycerol tribenzoate and glycerol phenylbutyrate together with a pharmaceutically acceptable carrier or excipient.

24. The process of claim 23, wherein the urea cycle disorder is selected from the group consisting of N-acetylglutamate synthase (NAGS) deficiency, Carbamoyl Phosphate Synthetase 1 (CPS1) deficiency, Ornithine transcarbamoylase (OTC) deficiency, Argininosuccinate synthase (ASS) deficiency, argininosuccinate lyase (ASL) deficiency, Arginase 1 (ARG1) deficiency, and any combination thereof.

25. The process of claim 23, wherein the pharmaceutical composition comprises from about 20 wt. % to about 99 wt. % glycerol tribenzoate and from about 20 wt. % to about 99 wt. % glycerol phenylbutyrate.
